(19) United States
(12) Patent Application Publication
(10) Pub. No.: US 2026/0157870 A1
(43) Pub. Date: Jun. 11, 2026

(54) Stent Delivery Systems

(71) Applicant: BOSTON SCIENTIFIC SCIMED, INC., MAPLE GROVE, MN (US)

(72) Inventors: Amanda Grace Stull, Maple Grove, MN (US); Brady Scott Logan, Monticello, MN (US); Michael William Nagel, Andover, MN (US)

(73) Assignee: BOSTON SCIENTIFIC SCIMED, INC., MAPLE GROVE, MN (US)

(21) Appl. No.: 19/364,129

(22) Filed: Oct. 21, 2025

Related U.S. Application Data
(60) Provisional application No. 63/710,329, filed on Oct. 22, 2024.

(51) Int. Cl. A61F 2/966

(52) U.S. Cl. CPC A61F 2/966

(57) ABSTRACT

Stent delivery systems as well as methods for making and using stent delivery systems are disclosed. An example stent delivery system may include an elongate shaft including an inner member having a stent receiving region, a deployment sheath slidably disposed along the inner member, and an intermediate shaft disposed between the inner member and the deployment sheath. A cuff may be coupled to the deployment sheath. The deployment sheath may include a floating braid region mechanically engaged with the cuff.

### CROSS-REFERENCE TO RELATED APPLICATION

This application claims the benefit of U.S. Patent Application Serial No. 63/710,329, filed October 22, 2024, entitled "STENT DELIVERY SYSTEMS”, which is incorporated by reference herein in its entirety.

### TECHNICAL FIELD

The present disclosure pertains to medical devices, and methods for manufacturing medical devices. More particularly, the present disclosure pertains to stent delivery systems.

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A stent delivery system is disclosed. The stent delivery system comprises: an elongate shaft including an inner member having a stent receiving region, a deployment sheath slidably disposed along the inner member, an intermediate shaft disposed between the inner member and the deployment sheath; and a cuff coupled to the deployment sheath; wherein the deployment sheath includes a floating braid region mechanically engaged with the cuff.

Alternatively or additionally to any of the embodiments above, the deployment sheath is coupled to a handle.

Alternatively or additionally to any of the embodiments above, the deployment sheath is rotatable relative to the handle.

Alternatively or additionally to any of the embodiments above, the deployment sheath includes a braided reinforcing member.

Alternatively or additionally to any of the embodiments above, the floating braid region is defined along a section of the braided reinforcing member.

Alternatively or additionally to any of the embodiments above, the cuff is thermally bonded to the deployment sheath.

Alternatively or additionally to any of the embodiments above, the floating braid region is mechanically bonded with the cuff.

Alternatively or additionally to any of the embodiments above, the cuff includes a surface texture.

Alternatively or additionally to any of the embodiments above, the cuff includes a single layer.

Alternatively or additionally to any of the embodiments above, the cuff includes a plurality of layers.

Alternatively or additionally to any of the embodiments above, the plurality of layers includes an inner layer and an outer layer different from the inner layer.

Alternatively or additionally to any of the embodiments above, the outer layer includes a lubricious material.

Alternatively or additionally to any of the embodiments above, the outer layer includes silicone.

Alternatively or additionally to any of the embodiments above, the cuff includes a tapered end region.

A stent delivery system is disclosed. The stent delivery system comprises: an elongate shaft including an inner member having a stent receiving region, a deployment sheath slidably disposed along the inner member; a handle coupled to the elongate shaft; wherein the deployment sheath is rotatable relative to the handle; a cuff bonded to the deployment sheath; wherein the deployment sheath includes a braid; and wherein a floating braid region is defined along the braid, the floating braid region being disposed at least partially within the cuff.

Alternatively or additionally to any of the embodiments above, the cuff includes a surface texture.

Alternatively or additionally to any of the embodiments above, the cuff includes a plurality of layers.

Alternatively or additionally to any of the embodiments above, the plurality of layers includes an inner layer and an outer layer different from the inner layer.

Alternatively or additionally to any of the embodiments above, the outer layer includes a lubricious material.

Alternatively or additionally to any of the embodiments above, the outer layer includes silicone.

A method for manufacturing a stent delivery system is disclosed. The method comprises disposing a cuff along an end region of a deployment sheath; wherein the deployment sheath includes a braid; thermally bonding the cuff to the deployment sheath without restraining the end region of the deployment sheath; and wherein thermally bonding the cuff to the deployment sheath defines a floating braid region that extends at least partially within the cuff.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is side view of an example system.

FIG. 2 is a side view of a portion of an example system.

FIG. 3 is a partial cross-sectional side view of a portion of an example system.

FIG. 4 is a partial cross-sectional side view of a portion of an example system.

FIG. 5 is a partial cross-sectional side view of a portion of an example system.

FIG. 6 is a side view of a portion of an example system.

FIG. 7 is a perspective view of a portion of an example system.

FIG. 8 is a side view of a portion of an example system.

FIG. 9 is a side view of a portion of an example system.

FIG. 10 is a side view of a portion of an example system.

FIG. 11 is a side view of a portion of an example system.

FIG. 12 is a side view of a portion of an example system.

FIG. 13 is a side view of a portion of an example system.

FIG. 14 is a side view of a portion of an example system.

FIG. 15 is a side view of a portion of an example system.

FIG. 16 is a side view of a portion of an example system.

FIG. 17 is a side view of a portion of an example system.

FIG. 18 is a side view of a portion of an example system.

FIG. 19 is a side view of a portion of an example system.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

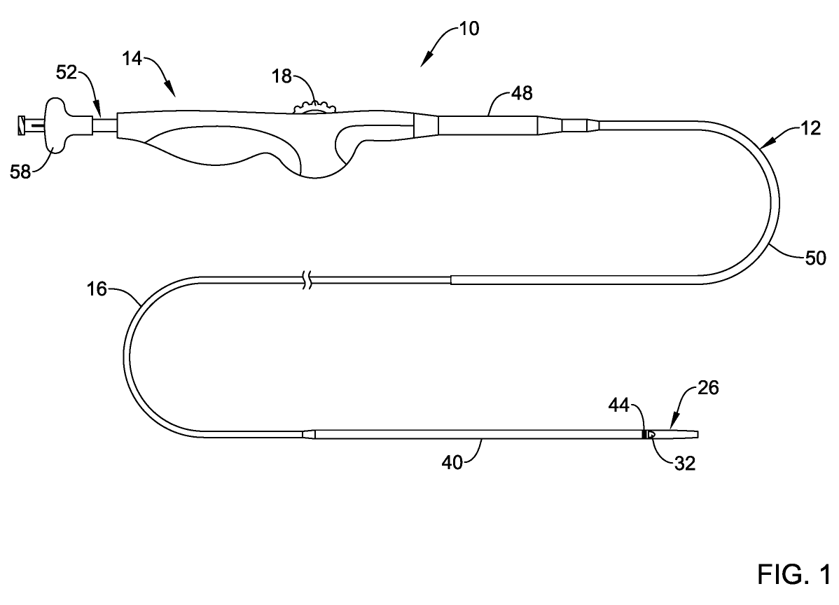

FIG. 1 illustrates an example stent delivery system 10. The system 10 may include a handle 14 coupled to an elongate shaft 12. In general, the system 10 may be used to deliver a suitable stent, graft, endoprosthesis, and/or the like to an area of interest within a body lumen of a patient. The body lumen may be a blood vessel located near the heart (e.g., within or near a cardiac vessel), within a peripheral vessel, within a neurological vessel, or at any other suitable location. Deployment of the stent may include the proximal retraction of a deployment sheath 16, which overlies the stent. Retraction of the deployment sheath 16 may include the actuation of an actuation member 18 generally disposed at the handle 14. In the example illustrated in FIG. 1, the actuation member 18 is a thumbwheel that can be rotated by a clinician in order to accomplish proximal retraction of the deployment sheath 16. Numerous other actuation members are contemplated. A number of other structures and features of the system 10 can be seen in FIG. 1 and are labeled with reference numbers. Additional discussion of some of these structures can be found below.

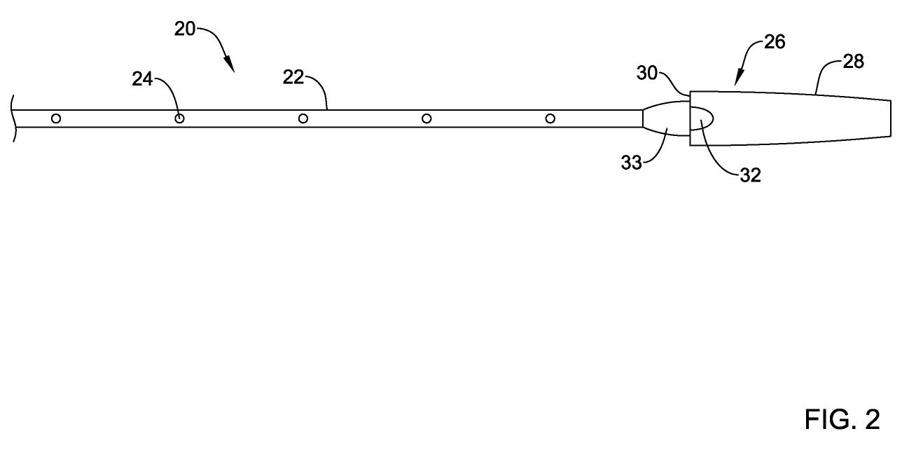

FIGS. 2-6 illustrate examples of some of the structural components that may be included as a part of the system 10. For example, the system 10 may include an inner shaft or member 20 as illustrated in FIG. 2. In at least some embodiments, the inner member 20 may be a tubular structure and, thus, may include a lumen (not shown). The lumen may be a guidewire lumen that extends along at least a portion of the length of the inner member 20. Accordingly, the system 10 may be advanced over a guidewire to the desired target location in the vasculature. In addition, or in alternative embodiments, the lumen may be a perfusion/aspiration lumen that allows portions, components, or all of the system 10 to be flushed, perfused, aspirated, or the like.

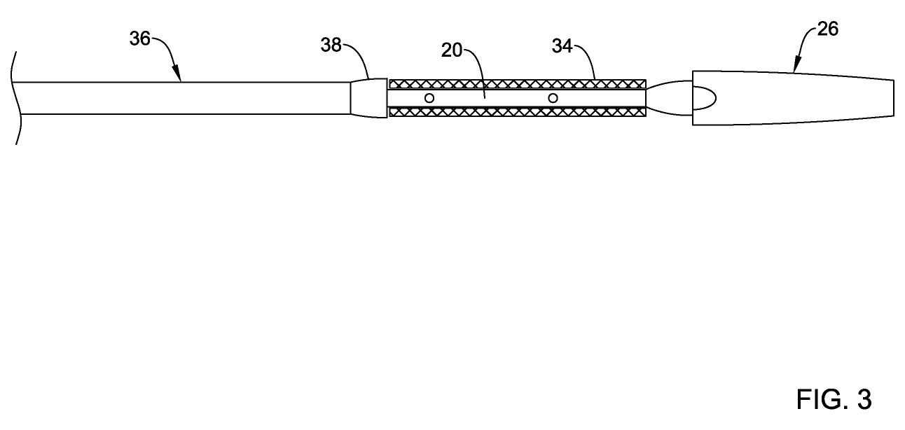

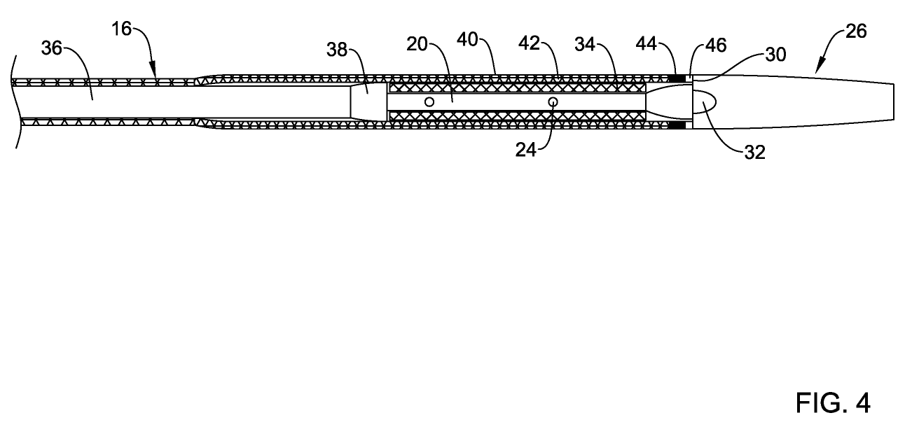

The inner member 20 may include a stent receiving region 22 about which a stent (not shown, can be seen in FIGS. 3-4) may be disposed. The length and/or configuration of the stent receiving region 22 may vary. For example, the stent receiving region 22 may have a length sufficient for the stent to be disposed thereon. It can be appreciated that as the length of the stent utilized for the system 10 increases, the length of the stent receiving region 22 also increases.

Along or otherwise disposed adjacent the stent receiving region 22 may be one or more perfusion ports 24. The ports 24 may extend through the wall of the inner member 20 such that fluid may be infused through the lumen of the inner member 20 and may be flushed through the ports 24. This may be desirable for a number of reasons. For example, the ports 24 may allow a clinician to evacuate air bubbles that may be trapped adjacent the stent by perfusing fluid through the ports 24. In addition, the ports 24 may be used to aspirate fluid that may be disposed along the inner member 20. The ports 24 may also aid in sterilization and/or other preparatory processing steps that may be involved in preparing the system 10 for use.

A tip 26 may be attached to or otherwise disposed at the distal end of the inner member 20. The tip 26 may generally have a rounded or smooth shape that provides a generally atraumatic distal end to the system 10. For example, the tip 26 may have a smooth tapered distal portion 28 that gently tapers. The tip 26 may also include a proximal ridge 30 that is configured so that the deployment sheath 16 can abut therewith. The tip 26 may also include a tapered proximal portion 33. In some instances, the tapered proximal portion 33 may aid the seating of the tip 26 in the deployment sheath 16. Numerous other shapes and/or configurations are contemplated for the tip 26.

The tip 26 may also include one or more cutouts or flats 32 formed therein. For the purposes of this disclosure, the flats 32 are understood to be cutouts or flattened portions of the tip 26 where the outer dimension or profile of the tip 26 is reduced. The name “flats” comes from the fact that these regions may have a somewhat “flat” appearance when compared to the remainder of the tip 26, which generally may have a rounded profile. The shape, however, of the flats 32 is not meant to be limited to being flat or planar as numerous shapes are contemplated.

The flats 32 may allow for a gap or space to be defined between the inner member 20 and the deployment sheath 16 when the deployment sheath 16 abuts the proximal ridge 30 of the tip 26. This gap may allow for fluid, for example perfusion fluid passed through the ports 24, to flow out from the deployment sheath 16. Thus, the flats 32 may be used in conjunction with the ports 24 to allow portions or all of the system 10 to be flushed or otherwise evacuated of air bubbles. One or more flats 32 may be positioned on the tip 26. For example, the tip 26 may have flats 32 positioned on opposite sides or 180 degrees from one another. In another example, the tip 26 may have flats 32 positioned circumferentially an equal distance or 90 degrees from one another.

FIG. 3 illustrates the inner member 20 with some additional structure of the system 10. In this figure, a stent 34 is disposed about the inner member 20 (e.g., about the stent receiving region 22 of the inner member 20). In some embodiments, the stent 34 is a self-expanding stent. Accordingly, the stent 34 may be biased to outwardly expand. Because of this, stent 34 may not be “loaded onto” the inner member 20 in a strict sense but rather may be thought of as being disposed about or surrounding the inner member 20. The stent 34 may then be restrained within the deployment sheath 16. In alternative embodiments, however, the stent 34 may be directly loaded onto the inner member 20 via crimping or any other suitable mechanical holding mechanism.

An intermediate tube 36 may also be disposed over the inner member 20. In at least some instances, the intermediate tube 36 may extend from a position adjacent to the proximal end of the inner member 20 to a position proximal of the distal end of the inner member 20. The intermediate tube 36 may include a bumper 38. In practice, the bumper 38 may function by preventing any unwanted proximal movement of the stent 34 during navigation and/or deployment of the stent 34.

The bumper 38 may have any suitable form. In some instances, the bumper 38 may be defined by a relatively short tube or sleeve that is disposed about the intermediate tube 36. The material utilized for the sleeve may be the same or different from that of the intermediate tube 36. The intermediate tube 36 may have a tapered or otherwise smooth transition in outer diameter adjacent the bumper 38. For example, polymeric material may be disposed or reflowed adjacent the bumper 38 (which may include disposing the polymeric material about a portion or all of the bumper 38) so as to define a gentle transition in outer diameter at the bumper 38. Other configurations are contemplated and may be utilized in alternative embodiments.

FIG. 4 illustrates additional structure of the system 10. Here the deployment sheath 16 can be seen disposed over the inner member 20, the intermediate tube 36, and the stent 34. It can be appreciated that the deployment sheath 16 is configured to shift between a first position, for example as shown in FIG. 4, where the deployment sheath 16 overlies the stent 34 and a second position where the deployment sheath 16 is proximally retracted to a position substantially proximal of the stent 34. In general, the first position may be utilized during navigation of the system 10 to the appropriate location within a body lumen and the second position may be used to deploy the stent 34.

The deployment sheath 16 may include a flared portion 40 where the outer diameter of the deployment sheath 16 is increased. In the flared portion 40, the thickness of the tubular wall of the deployment sheath 16 may or may not be increased. The flared portion may allow the deployment sheath 16 to have an adequate inner dimension that is suitable so that deployment sheath 16 may be disposed about the stent 34 and the bumper 38.

In at least some instances, the deployment sheath 16 may include a reinforcing member 42 embedded or otherwise included therewith. The reinforcing member 42 may have any number of a variety of different configurations. For example, the reinforcing member 42 may include a braid, coil, mesh, combinations thereof, or the like, or any other suitable configuration. In some instances, the reinforcing member 42 may extend along the entire length of the deployment sheath 16. In other instances, the reinforcing member 42 may extend along one or more portions of the length of the deployment sheath 16. For example, the reinforcing member 42 may extend along the flared portion 40.

The deployment sheath 16 may also include a radiopaque marker or band 44. In general, the marker band 44 may be disposed adjacent to the distal end 46 of the deployment sheath 16. One or more additional marker bands 44 may be disposed along other portions of the deployment sheath 16 or other portions of the system 10. The marker band 44 may allow the distal end 46 of the deployment sheath 16 to be fluoroscopically visualized during advancement of the system 10 and/or deployment of the stent 34.

FIG. 4 also illustrates the distal end 46 of the deployment sheath 16 abutting the proximal ridge 30. In this configuration, the stent 34 can be flushed (e.g., to remove air bubbles) by infusing fluid through the inner member 20 and through the ports 24. Because of the flats 32, fluid may be allowed to be flushed out of the deployment sheath 16 by passing through the gaps formed between the inner member 20 and the deployment sheath 16 at the flats 32.

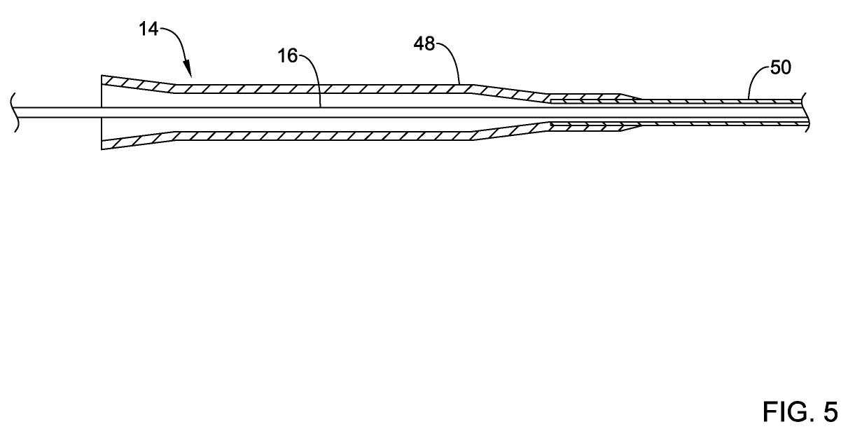

FIG. 5 illustrates a distal portion 48 of the handle 14. Here it can be seen that the handle 14 is attached to an outer member or shaft 50. The outer shaft 50 may be disposed about the deployment sheath 16 and extends along a portion of the length of the deployment sheath 16. Thus, along at least a portion of the length of the system 10, the system 10 may include four tubular structures that may be coaxially arranged – namely the outer shaft 50, the deployment sheath 16, the intermediate tube 36, and the inner member 20. In at least some embodiments, the outer shaft 50 may provide the system 10 with a number of desirable benefits. For example, the outer shaft 50 may include or otherwise be formed from a lubricious material that can reduce friction that may be associated with proximally retracting the deployment sheath 16. In addition, the outer shaft 50 may comprise a surface that can be clamped or otherwise locked so that the position of the system 10 can be maintained without negatively impacting the retraction of the deployment sheath 16 (which might otherwise be impacted if the deployment sheath 16 was to be clamped). Numerous other desirable benefits may also be achieved through the use of the outer shaft 50.

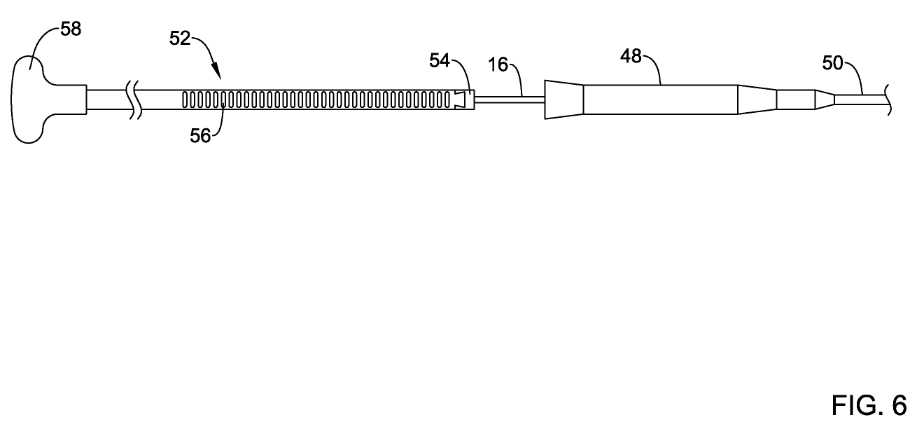

The deployment sheath 16 may pass proximally through the outer shaft 50 and extend proximally back within the handle 14. The intermediate tube 36 and the inner member 20 both also extend back within the handle 14 and are disposed within the deployment sheath 16. The proximal end of the deployment sheath 16 may be attached to a rack member 52 with a fastener or clip 54 as illustrated in FIG. 6. Thus, it can be appreciated that proximal movement of the rack member 52 may result in analogous proximal movement of the deployment sheath 16. The rack member 52 may include a plurality of teeth or gears 56. In practice, the teeth 56 may be configured to engage with corresponding teeth or gears (not shown) on the thumbwheel 18. Consequently, rotation of the thumbwheel 18, via gearing thereof with the gears 56, can be utilized to proximally retract the rack member 52 and, thus, the deployment sheath 16. Other structural arrangements may be utilized to accomplish proximal retraction of the rack member 52 through the actuation of the thumbwheel 18 or any other suitable actuation member.

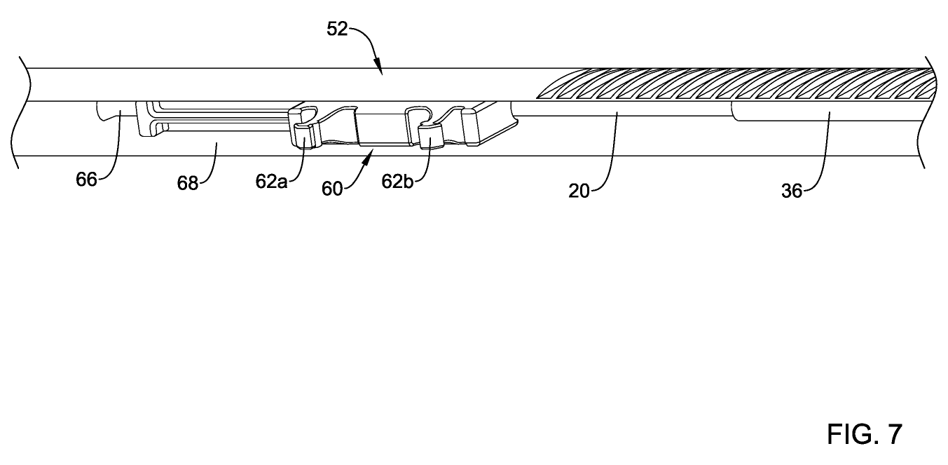

A pull grip 58 may be coupled to the rack member 52. When properly assembled, the main body of the rack member 52 may be disposed within handle 14 and the pull grip 58 may be disposed along the exterior of the handle 14. The rack member 52 may have a slot or groove 68 formed therein (not shown in FIG. 6, can be seen in FIG. 7). The slot 68 may extend the length of the rack member 52, including extending along the pull grip 58. Because the pull grip 58 may be generally located near the proximal end of the inner member 20, the flared shape of the pull grip 58 and the orientation of the slot 68 may allow the pull grip 58 to function as a guidewire introducer or funnel that may assist a clinician in placing, holding, removing, and/or exchanging a guidewire extending through the inner member 20.

In order to properly deploy the stent 34, the various components of the system 10 may need to work in concert so that relative motion of the deployment sheath 16 can be accomplished relative to the inner member 20. In addition, to improve the accuracy of deployment, the intermediate tube 36 may need to be configured so as to provide the desired longitudinal support necessary to limit proximal movement of the stent 34. In at least some embodiments, the proper configuration of these structures may be maintained, at least in part, through the use of a clip member 60 as illustrated in FIG. 7.

In general, the clip member 60 is disposed within the handle 14 and is configured to be secured along the interior of the handle 14. Accordingly, the clip member 60 allows the longitudinal position of one or more portions of the system 10 to be fixed relative to the handle 14. In order to secure the clip member 60 to the handle 14, the clip member 60 may include one or more fasteners or legs 62a/62b. For example, the handle 14 may have one or more slots, grooves, openings, or the like that are configured to seat the legs 62a/62b such that the relative position of the clip member 60 relative to the handle 14 is fixed. In some embodiments, the clip member 60 may be configured to “snap in” to the handle 14. This may desirably simplify manufacturing.

The orientation of the clip member 60 may be such that it is positioned near one or more structures of the system 10. In at least some embodiments, the clip member 60 may be configured so that at least a portion thereof is positioned within the slot 68 of the rack member 52. This may desirably place the clip member 60 near the inner member 20 and the intermediate tube 36 (which may also extend through the slot 68) such that the clip member 60 can be associated therewith. As such, the clip member 60 may aid in maintaining the relative position of one or more structures of the system 10 so that the stent 34 can be accurately deployed. For example, the clip member 60 may include one or more tubular portions that the inner member 20 may pass through and the inner member 20 may optionally include a flared proximal end 66 that may substantially prevent the inner member 20 from moving distally beyond the tubular portion(s). In some embodiments, the clip member 60 may include a flared region or end (not shown), which may facilitate entry of a guidewire into the clip member 60 and/or the inner member 20.

When the stent 34 is deployed, a clinician may actuate the actuation thumbwheel 18 (FIG. 1). Because of the association of the thumbwheel 18 with the rack member 52, relative rotation of the thumbwheel 18 causes proximal movement of the deployment sheath 16. As the deployment sheath 16 proximally retracts, the stent 34 is “uncovered” and (if the stent 34 is a self-expanding stent) can expand within the body lumen.

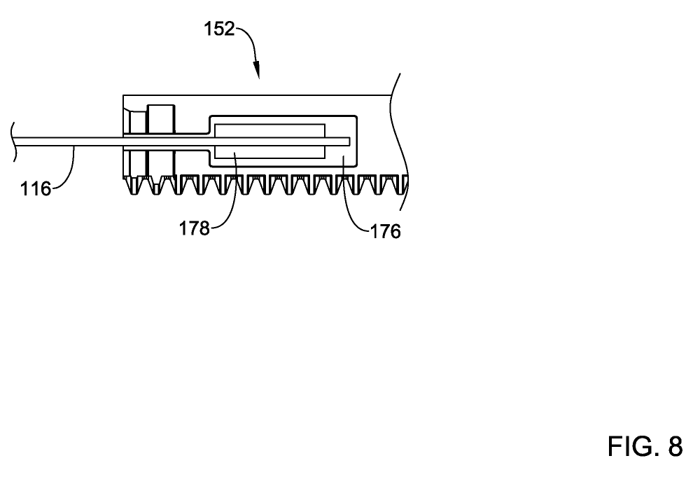

FIG. 8 illustrates an example deployment sheath 116 (e.g., similar in form and function to other deployment sheaths disclosed herein) secured to an example rack 152 (e.g., similar in form and function to other racks disclosed herein). In this example, the rack 152 may include a sidewall opening 176. The sidewall opening 176 may be configured to allow the deployment sheath 116 to be mechanically secured to the rack 152. For example, the deployment sheath 116 may be threaded through the distal end of the rack 152 and then through the sidewall opening 176. If desired, a structure such as a cuff or sleeve 178 maybe bonded to the deployment sheath 116. Bonding the cuff 178 to the deployment sheath 116 may include a suitable bonding process such as thermal bonding, adhesive bonding, mechanically bonding, combinations thereof, and/or the like. The deployment sheath 116 may then be advanced until the cuff 178 is seated within the sidewall opening 176 in a manner that axially secures the deployment sheath 116 relative to the rack 152 while permitting the deployment sheath 116 to rotate relative to the rack 152. Some example structures for mechanically securing the deployment sheath 116 with the rack 152 may include those disclosed in U.S. Patent No. 11,602,447, the entire disclosure of which is herein incorporated by reference.

Tension forces may tend to build up or otherwise be present in the deployment sheath 116, for example during delivery of the stent 34. Because of this, it may be desirable for the cuff 178 to be secured to the deployment sheath 116 in a manner that can withstand forces, for example tension forces on the deployment sheath 116. Additionally, the rotational freedom of the deployment sheath 116 can help pullback efficiency (e.g., which may be understood to be analyzing a ratio of the maximum pullback force to the stent pullout force). It may be desirable to increase the pullback efficiency. Disclosed herein are systems where the cuff 178 is secured in a manner that can withstand elevated forces and methods for securing the cuff 178 in a such a manner. Additionally, disclosed herein are systems where pullback efficiency is improved.

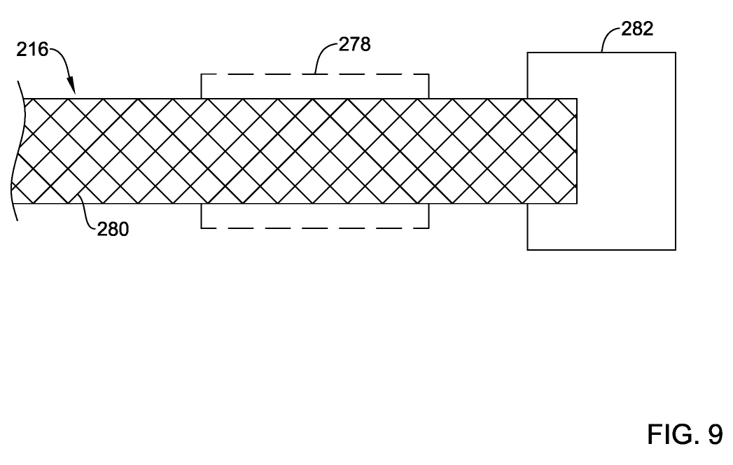

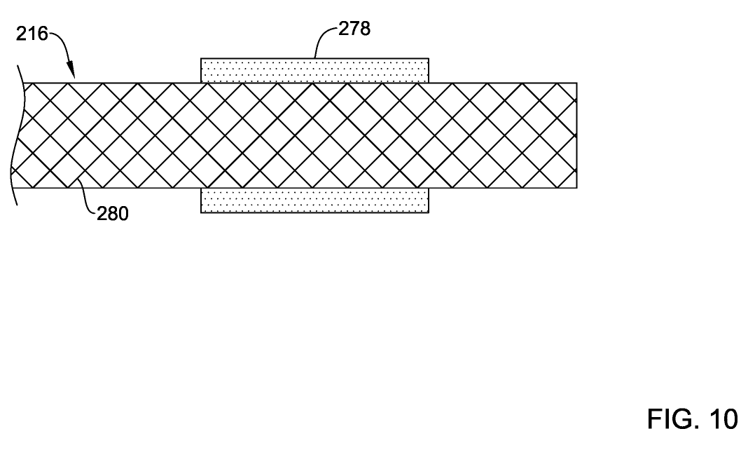

FIG. 9 illustrates an example deployment sheath 216 that may be similar in form and function to other deployment sheaths disclosed herein. In this example, the deployment sheath 216 may include a braid or braided support member 280. The braid 280 may be disposed along the outer surface of the deployment sheath 216. In at least some instances, an outer sleeve or coating may be disposed along the braid 280. It can be appreciated that the braid 280 may have a level of stress therein, for example due to forces being applied to the wires of the braid 280 while applying the braid 280 the deployment sheath 216. If a fixture, schematically illustrated in FIG. 9 as a fixture 282, is used to secure an end of the deployment sheath 216 while bonding/securing a cuff 278 thereto, the stresses in the braid 280 may tend to hold the braid 280 in place. Because of this, the braid 280 may generally stay in position and not migrate toward/into the cuff 278 when the cuff 278 is bonded to the deployment sheath 216 as shown in FIG. 10.

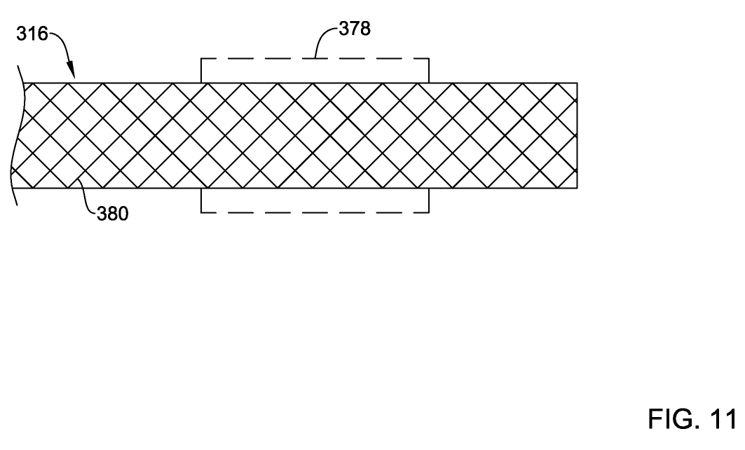

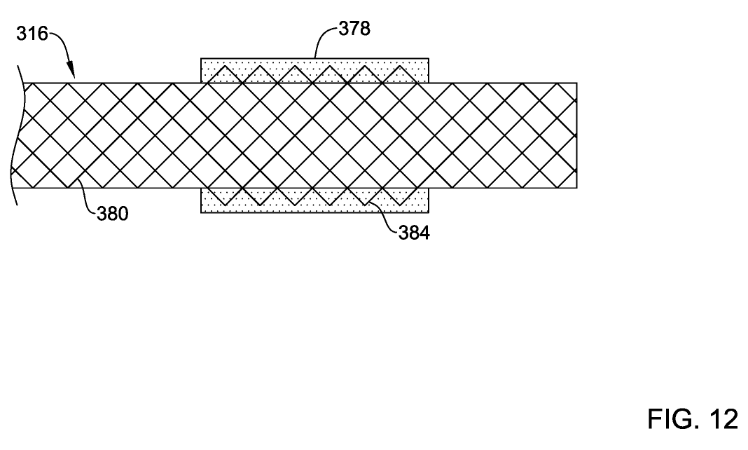

FIG. 11 illustrates another example deployment sheath 316 that may be similar in form and function to other deployment sheaths disclosed herein. The deployment sheath 316 may include a braid or braided support member 380. In this example, an end region of the deployment sheath 316 may be “free” (e.g., not be secured or held by a fixture such as the fixture 282) when bonding a cuff 378 thereto. Because the end region of the deployment sheath 316 is free during bonding of the cuff 378, the braid 380 may have the ability for some of the stresses therein to be released/relieved, thereby creating or defining a floating braid region 384 that can expand into the cuff 378 as depicted in FIG. 12. The floating braid region 384 may be understood to be a portion of the braid 380 where the braid 380 can expand radially outward, for example during a bonding process (e.g., a thermal bonding process) where the cuff 378 is bonded (e.g., thermally bonded) with the deployment sheath 316. The floating nature of the floating braid region 384 is akin to the braid 380 partially unwinding and radially expanding. The outwardly expanded portion of the braid 380, rather than being closely associated with the deployment sheath 316, expands radially outward or “floats” relative to the remainder of the braid 380 (e.g., which stays closely associated with the deployment sheath 316). Thus, the floating braid region 384 may extending radially outward further that the remainder of the braid 380.

The floating braid region 384 help to increase the robustness of the bond between the cuff 378 and the deployment sheath 316. For example, the floating braid region 384 may form a mechanical linkage, engagement, and/or bond with the cuff 378, which help to increase the strength and/or tension resistance of the bond (e.g., the thermal bond) between the cuff 378 and the deployment sheath 316. In other words, the floating braid region 384 may improve the thermal bond by forming a mechanical engagement and/or bond (e.g., in addition to the thermal bond). Because of this, the deployment sheath 316 and the cuff 378 are able to withstand higher forces, for example tensile force, during deployment of the stent 34. For example, the floating braid region 384 may add about 0.5-5 or more pounds per square inch (3447-34473 Pa), or about 1-3 or more pounds per square inch (6895-20684 Pa), or about 2 or more pounds per square inch (13790 Pa) of strength (e.g., tensile strength).

Additionally, the use of the cuff 378 for securing the deployment sheath 316 to the rack (e.g., the rack 52, 152) in a manner that permits rotation of the deployment sheath 316 relative to the rack (e.g., the rack 52, 152) has been shown to increase the pullback efficiency, which may be understood to be the ratio of the maximum pullback force applied to the deployment sheath 316 during deployment to the stent pullout force. For example, a ratio of the maximum pullback force applied to the deployment sheath 316 during deployment to the stent pullout force may be on the order of about 1.2:1 to 2.4:1, or about 1.5:1 to 2.1:1, or about 1.8:1.

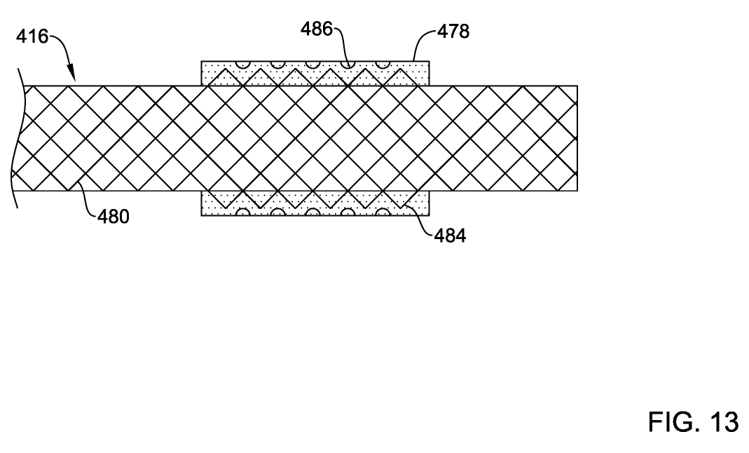

FIG. 13 illustrates another example deployment sheath 416 that may be similar in form and function to other deployment sheaths disclosed herein. The deployment sheath 416 may include a braid or braided support member 480. The braid 480 may include a floating braid region 484 that may migrate into a cuff 478. In some instances, the cuff 478 may include one or more structural features that help to reduce friction between the cuff 478 and the rack (e.g., the rack 52, 152). For example, the cuff 478 may include one or more surface textures 486 such as dimples or dimpled regions. The surface textures 486 may help to reduce the surface area of the cuff 478 contacting the rack (e.g., the rack 52, 152), which may help to reduce friction and/or improve rotatability of the deployment sheath 416 relative to the rack (e.g., the rack 52, 152). Forming the surface texture 486 may include a molding or casting process (e.g., where the mold may include surface texturing), etching (e.g., laser, chemical, mechanical, etc.), ablation, and/or the like.

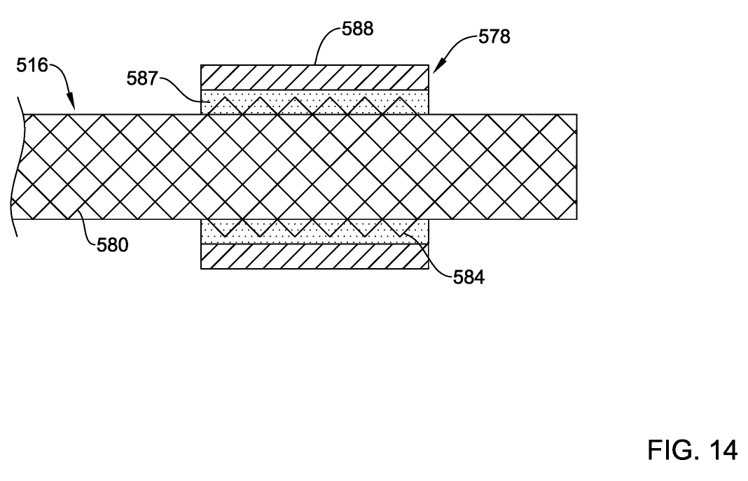

FIG. 14 illustrates another example deployment sheath 516 that may be similar in form and function to other deployment sheaths disclosed herein. The deployment sheath 516 may include a braid or braided support member 580. The braid 580 may include a floating braid region 584 that may migrate into a cuff 578. In some instances, the cuff 578 may include one or more (e.g., 2, 3, 4, 5, or more) layers and/or a coating. For example, the cuff 578 may include an inner or base layer 587 and an outer layer 588. In some instances, the inner layer 587 and the outer layer 588 may be formed from the same material. Alternatively, the inner layer 587 and the outer layer 588 may be formed from different materials. When formed from different materials, the outer layer 588 may have a different softening/melting point than the inner layer 587, which may help to control/limit the depth that the floating braid region 584 extends into the cuff 578. For example, the outer layer 588 may have a higher softening/melting point than the inner layer 587 so that the floating braid region 584 can migrate into/through the inner layer 587 (e.g., during a thermal bonding process) and the migration can be stopped at/by the outer layer 588. In some of these and in other instances, the outer layer 588 may take the form of a lubricious coating such as a silicone coating and/or the like.

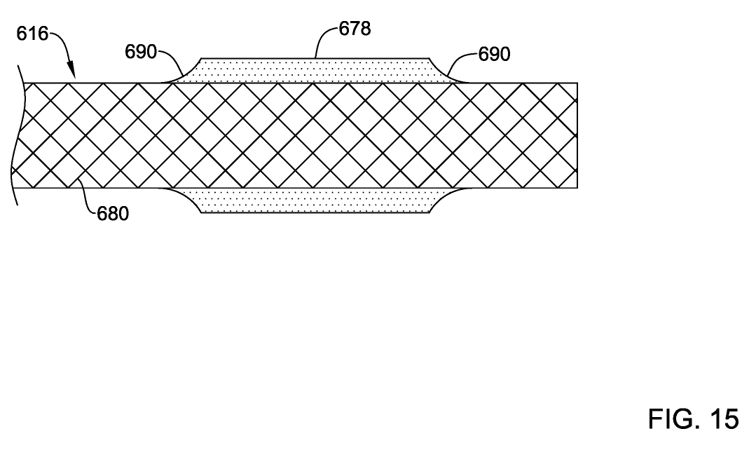

FIG. 15 illustrates another example deployment sheath 616 that may be similar in form and function to other deployment sheaths disclosed herein. The deployment sheath 616 may include a braid or braided support member 680. A cuff 678 may be secured to the deployment sheath 616. The cuff 678 may include tapered end regions 690 (e.g., the distal end region, the proximal end region, or both of the cuff 678 may be tapered). The tapered end regions 690 may help to bolster the strength and robustness of the cuff 678 and/or the bond between the cuff 678 and the deployment sheath 616.

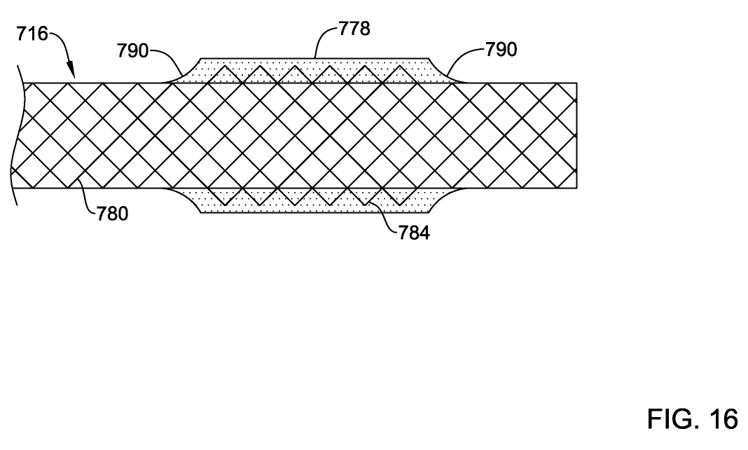

FIG. 16 illustrates another example deployment sheath 716 that may be similar in form and function to other deployment sheaths disclosed herein. The deployment sheath 716 may include a braid or braided support member 780. The braid 780 may include a floating braid region 784 that may migrate into a cuff 778. Like the cuff 678, the cuff 778 may include tapered end regions 790.

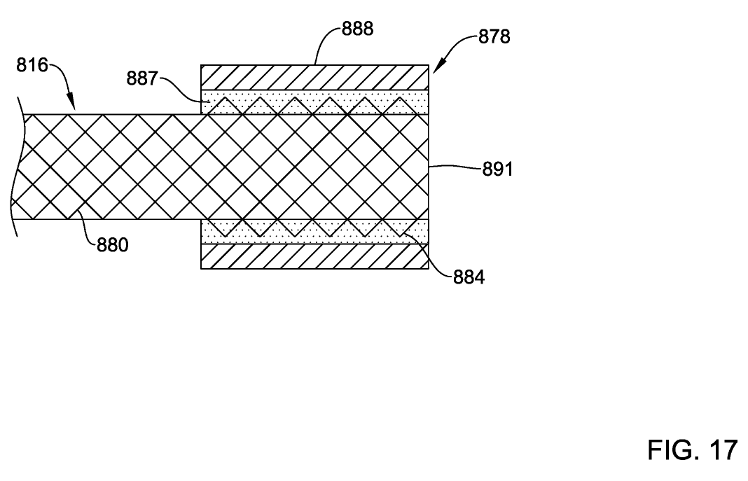

FIG. 17 illustrates another example deployment sheath 816 that may be similar in form and function to other deployment sheaths disclosed herein. The deployment sheath 816 may include a braid or braided support member 880. The braid 880 may include a floating braid region 884 that may migrate into a cuff 878. In some instances, the cuff 878 may include one or more (e.g., 2, 3, 4, 5, or more) layers and/or a coating. For example, the cuff 878 may include an inner or base layer 887 and an outer layer 888. In some instances, the inner layer 887 and the outer layer 888 may be formed from the same material. Alternatively, the inner layer 887 and the outer layer 888 may be formed from different materials. When formed from different materials, the outer layer 888 may have a different softening/melting point than the inner layer 887, which may help to control/limit the depth that the floating braid region 884 extends into the cuff 878. For example, the outer layer 888 may have a higher softening/melting point than the inner layer 887 so that the floating braid region 884 can migrate into/through the inner layer 887 (e.g., during a thermal bonding process) and the migration can be stopped at/by the outer layer 888. In some of these and in other instances, the outer layer 888 may take the form of a lubricious coating such as a silicone coating and/or the like.

In some instances, the cuff 878 may be disposed proximally of a distal end 891 of the deployment sheath 816. In other instances, the cuff 878 may extend to or otherwise be disposed at the distal end 891 of the deployment sheath 816 as depicted in FIG. 17. This may help to improve migration of the floating braid region 884 into the cuff 878. For example, the support member 880 may have a tendency to unwind/unravel adjacent to its end (e.g., adjacent to the distal end 891), which may help the floating braid region 884 migrate into the cuff 878. It can be appreciated that the arrangement where the cuff 878 is disposed at the distal 891 of the deployment sheath 816 may be utilized with any of the other deployment sheaths and/or cuffs disclosed herein, when appropriated.

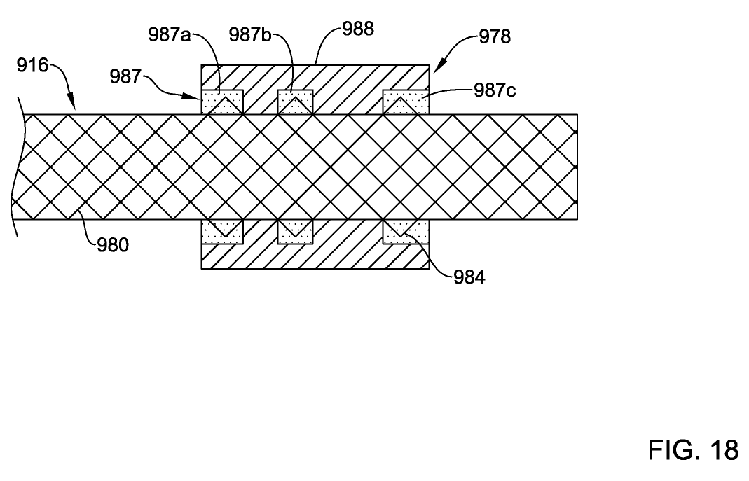

FIG. 18 illustrates another example deployment sheath 916 that may be similar in form and function to other deployment sheaths disclosed herein. The deployment sheath 916 may include a braid or braided support member 980. The braid 980 may include a floating braid region 984 that may migrate into a cuff 978. In some instances, the cuff 978 may include one or more (e.g., 2, 3, 4, 5, or more) layers and/or a coating. For example, the cuff 978 may include an inner or base layer 987 and an outer layer 988. In some instances, the inner layer 987 and the outer layer 988 may be formed from the same material. Alternatively, the inner layer 987 and the outer layer 988 may be formed from different materials. When formed from different materials, the outer layer 988 may have a different softening/melting point than the inner layer 987, which may help to control/limit the depth that the floating braid region 984 extends into the cuff 978. For example, the outer layer 988 may have a higher softening/melting point than the inner layer 987 so that the floating braid region 984 can migrate into/through the inner layer 987 (e.g., during a thermal bonding process) and the migration can be stopped at/by the outer layer 988. In some of these and in other instances, the outer layer 988 may take the form of a lubricious coating such as a silicone coating and/or the like.

In the example shown in FIG. 18, the inner layer 987 may be formed as a plurality of sections or regions such as regions 987a, 987b, 987c. In some instances, the regions 987a, 987b, 987c may be discrete and/or axially spaced apart. Alternatively, one or more of the regions 987a, 987b, 987c may be connected or otherwise continuous with one another. The regions 987a, 987b, 987c of the inner layer 987 may allow for the floating braid region 984 to migrate therein. The arrangement of the regions 987a, 987b, 987c may form gaps or spaces in the floating braid region 984, which may help to improve the integrity of the bond between the cuff 978 and the deployment sheath 916.

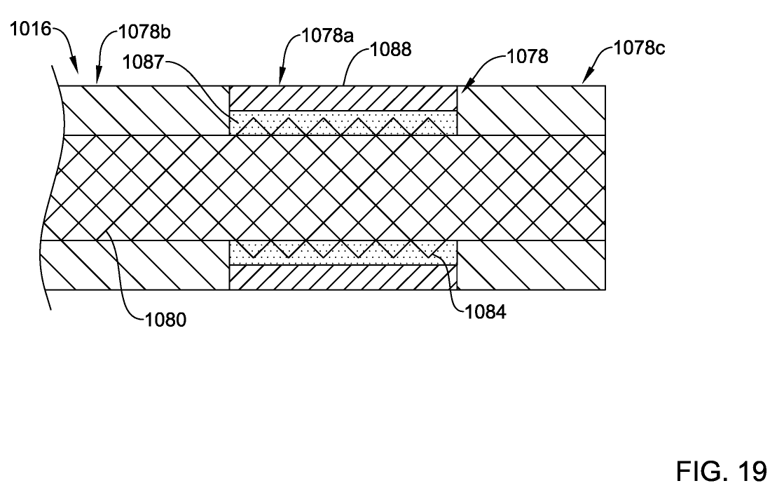

FIG. 19 illustrates another example deployment sheath 1016 that may be similar in form and function to other deployment sheaths disclosed herein. The deployment sheath 1016 may include a braid or braided support member 1080. The braid 1080 may include a floating braid region 1084 that may migrate into a cuff 1078. In some instances, the cuff 1078 may include one or more sections such as sections 1078a, 1078b, 1078c. Section 1078a of the cuff 1078 include one or more (e.g., 2, 3, 4, 5, or more) layers and/or a coating. For example, section 1078a of the cuff 1078 may include an inner or base layer 1087 and an outer layer 1088. In some instances, the inner layer 1087 and the outer layer 1088 may be formed from the same material. Alternatively, the inner layer 1087 and the outer layer 1088 may be formed from different materials. When formed from different materials, the outer layer 1088 may have a different softening/melting point than the inner layer 1087, which may help to control/limit the depth that the floating braid region 1084 extends into the cuff 1078. For example, the outer layer 1088 may have a higher softening/melting point than the inner layer 1087 so that the floating braid region 1084 can migrate into/through the inner layer 1087 (e.g., during a thermal bonding process) and the migration can be stopped at/by the outer layer 1088. In some of these and in other instances, the outer layer 1088 may take the form of a lubricious coating such as a silicone coating and/or the like.

Section 1078b and/or section 1078c may be disposed on flanking ends of the section 1078a. In at least some instances, the sections 1078b, 1078c may be formed from a material similar to or the same as the outer layer 1088. For example, the sections 1078b, 1078c may include a material with a higher softening/melting point than the inner layer 1087 so that the sections 1078b, 1078c may tend to resist migration of the support member 1080 therein.

The materials that can be used for the various components of the system 10 may include those commonly associated with medical devices. For simplicity purposes, the following discussion makes reference to the shaft 12 and other components of the system 10. However, this is not intended to limit the devices and methods described herein, as the discussion may be applied to other similar tubular members and/or components of tubular members or devices disclosed herein.

The shaft 12 and/or other components of the system 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the system 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the system 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system 10 to achieve the same result.

In some embodiments, a degree of Magnetic Resonance Imaging (MRI) compatibility is imparted into the system 10. For example, the system 10, or portions thereof, may be made of a material that does not substantially distort the image and create substantial artifacts (e.g., gaps in the image). Certain ferromagnetic materials, for example, may not be suitable because they may create artifacts in an MRI image. The system 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A stent delivery system, comprising:
an elongate shaft including an inner member having a stent receiving region,
a deployment sheath slidably disposed along the inner member,
an intermediate shaft disposed between the inner member and the deployment sheath; and
a cuff coupled to the deployment sheath;
wherein the deployment sheath includes a floating braid region mechanically engaged with the cuff.

2. The stent delivery system of claim 1, wherein the deployment sheath is coupled to a handle.

3. The stent delivery system of claim 2, wherein the deployment sheath is rotatable relative to the handle.

4. The stent delivery system of claim 1, wherein the deployment sheath includes a braided reinforcing member.

5. The stent delivery system of claim 4, wherein the floating braid region is defined along a section of the braided reinforcing member.

6. The stent delivery system of claim 1, wherein the cuff is thermally bonded to the deployment sheath.

7. The stent delivery system of claim 6, wherein the floating braid region is mechanically bonded with the cuff.

8. The stent delivery system of claim 1, wherein the cuff includes a surface texture.

9. The stent delivery system of claim 1, wherein the cuff includes a single layer.

10. The stent delivery system of claim 1, wherein the cuff includes a plurality of layers.

11. The stent delivery system of claim 10, wherein the plurality of layers includes an inner layer and an outer layer different from the inner layer.

12. The stent delivery system of claim 11, wherein the outer layer includes a lubricious material.

13. The stent delivery system of claim 11, wherein the outer layer includes silicone.

14. A stent delivery system, comprising:
an elongate shaft including an inner member having a stent receiving region,
a deployment sheath slidably disposed along the inner member;
a handle coupled to the elongate shaft;
wherein the deployment sheath is rotatable relative to the handle;
a cuff bonded to the deployment sheath;
wherein the deployment sheath includes a braid; and
wherein a floating braid region is defined along the braid, the floating braid region being disposed at least partially within the cuff.

15. The stent delivery system of claim 14, wherein the cuff includes a surface texture.

16. The stent delivery system of claim 14, wherein the cuff includes a plurality of layers.

17. The stent delivery system of claim 16, wherein the plurality of layers includes an inner layer and an outer layer different from the inner layer.

18. The stent delivery system of claim 17, wherein the outer layer includes a lubricious material.

19. The stent delivery system of claim 17, wherein the outer layer includes silicone.

20. A method for manufacturing a stent delivery system, the method comprising:
disposing a cuff along an end region of a deployment sheath;
wherein the deployment sheath includes a braid;
thermally bonding the cuff to the deployment sheath without restraining the end region of the deployment sheath; and
wherein thermally bonding the cuff to the deployment sheath defines a floating braid region that extends at least partially within the cuff.
